(19) United States
(12) Patent Application Publication
(10) Pub. No.: US 2007/0055289 A1
(43) Pub. Date: Mar. 8, 2007

(54) Digital stereotaxic manipulator with controlled angular displacement and fine-drive mechanism

(76) Inventors: Charles W. Scouten, Downers Grove, IL (US); James G. Unnerstall, O'Fallon, MO (US); John M. Thompson, Troy, IL (US)

Correspondence Address: Patrick D. Kelly, 11939 Manchester #403, St. Louis, MO, 63131, US

(21) Appl. No.: 10/636,899

(22) Filed: Aug. 6, 2003

(51) Int. Cl. A61B 19/00

(52) U.S. Cl. 606/130

(57) ABSTRACT

A digital stereotaxic manipulator for animal research is provided with two enhancements. In one enhancement, rotary encoders are provided, to allow tilting of the manipulator, about vertical and/or horizontal axes, to be measured within a fraction of a degree. With assistance from software that applies sine and cosine values, orthogonal coordinates that are emitted by linear reader heads can be corrected to provide accurate orthogonal coordinates, even when an instrument has been rotated and/or tilted substantially. In a second enhancement, a “fine-drive” mechanism provides precise control over dorsal/ventral motion of an instrument. A radial gear is mounted on the main threaded shaft in the vertical arm assembly. A helical gear on a horizontal shaft is used to drive rotation of the radial gear and vertical shaft. If a 1:20 gearing ratio is provided by the helical and radial gears, this provides an operator with 20-times more precise control over vertical motion of an instrument. A detente is also provided, to enable the helical gear to pop out of position without damage, if an operator rotates the main vertical shaft before disengaging the fine-drive mechanism. A digital display device with touch-screen controls is also disclosed.

### BACKGROUND OF THE INVENTION

This invention relates to equipment used in biological and medical research that uses small animals, such as rats or mice.

Numerous types of biological and medical research require that the head and/or spine of a rat, mouse, or other small animal must be held in a secure and stationary position throughout the duration of a surgical, drug injection, or similar procedure. One major category of such research, which is discussed throughout this text as an example but which is not intended to be limiting, involves invasive neurological procedures carried out on rats or mice, which are widely used in neurology research because of several factors (including low cost, ease of breeding and care, and the ready availability of extensive information on the gene sequences of both species). For convenience, the discussion below refers to rats as an exemplary species, but it should be understood that these comments and teachings also apply to other types of animals, including mammals such as rabbits, cats, and dogs, as well as various non-mammalian species such as frogs, toads, birds, and fish or other marine animals. Similarly, manipulator devices as disclosed herein (scaled up to larger sizes, if desired) can be bolted or otherwise affixed to a stereotaxic base having a U-frame and clamping components sized for larger animals, such as livestock.

The types of neurological research that can be greatly enhanced by the devices disclosed herein include, for example, implantation of a stimulatory or measuring electrode in a specific targeted region of the brain, or insertion of a microscopic needle that will be used to inject a test compound, genetic vector, genetically engineered cells, or stem cells into a targeted brain region. It should also be noted that various types of veterinary, breeding-related, and other therapeutic or otherwise useful treatments that involve the head and/or spinal cord are being developed for various types of animals, and the devices disclosed herein can be readily adapted to such uses, in ways that will be apparent to those skilled in the art.

When these types of procedures are performed, the head of the rat or other animal must be held in a totally immobilized position. To accomplish that type of immobilization, neurology researchers typically use devices called “stereotaxic holders” during an invasive procedure. These types of holders are commercially available from several companies, including myNeurolab, Inc. (St. Louis, Mo.; www.myNeurolab.com).

A typical stereotaxic holder as used in the prior art, designed for holding a rat or other small animal, is shown in FIG. 1. The main components of the lower portion, which is referred to herein as holder assembly 100, include a base plate 102, a U-frame 104, and several clamping components. The base plate 104 preferably should be large enough to completely support a rat's entire body, and heavy enough to minimize any motion if a worker inadvertently bumps or jostles it during a test. The “U-frame” 104 is supported at an appropriate height by sturdy posts, and during use, the animal's head is held securely in place by a combination of two slidable “ear pins” (also called ear bars) 110 and 112, and a snout clamp 121.

The holder assembly 100 holds an animal in a fixed position, and a manipulator assembly 200 (which is described in more detail below, and which normally is securely bolted to the holder assembly 100 during a procedure) is used to manipulate and move an instrument in a precise manner, to carry out an invasive procedure on the head or brain, spinal cord, or other anatomical structure. For convenience, any device (such as a needle, blade, electrode tip, patch clamp, etc.) that is moved with the assistance of a manipulator, and that actually touches an animal during a procedure, is referred to herein as an instrument.

For convenience, any manipulation or intervention involving an animal being held in a stereotaxic holder is referred to herein as a procedure. Although the word “test” is occasionally used by some to refer to such procedures, in many situations the actual test(s) that will evaluate the effects of a surgical, drug injection, or other procedure may not be carried out until days, weeks, or even months later.

The degree of control needed for invasive procedures on a rat or mouse are usually measured in either: (i) tenths of a millimeter, for procedures carried out with the naked eye; or, (ii) microns, for procedures carried out with the aid of a microscope. For reference, a micron is 1/1,000 of a millimeter. Most mammalian cells have diameters that are in the general range of about 10 microns, which is 1/100 of a millimeter. However, most types of neurons (and many types of glial cells) also have long fibrous extensions (which include axons, dendrites, and other types of “processes”) with diameters in the single-micron or sub-micron range; therefore, manipulator devices as disclosed herein that can provide single-micron or sub-micron degrees of accuracy can be highly useful, in neurological research.

Since unaided hands cannot provide the degree of precision and control that is needed for most types of invasive neurological procedures, manipulators are designed to control and move instruments in a more careful and precise manner than can be achieved by hand.

A typical manipulator 200 with a general design that has been known for decades, and that allows only manual readings (also referred to herein as “vernier” or “analog” readings) is shown in FIG. 1. It is bolted, in a detachable manner, to the U-frame 104 on stereotaxic holder 100.

The term “vernier” refers to a type of etched or printed visual device that can be marked on a rod or other physical structure, to help researchers visually interpret linear scales with greater accuracy (usually to within 0.1 millimeter accuracy). These devices are provided on every non-digital “old-style” stereotaxic manipulator sold for the last several decades. Therefore, stereotaxic manipulators that do not have electronic measuring or display capability, and that must be read visually by inspecting etched linear scales on the slide, the vertical arm, and the horizontal arm, are referred to herein as “vernier” systems.

As described in copending application Ser. No. 10/036,231, filed Dec. 24, 2001 (by Scouten et al, published and accessible on the U.S. PTO website under accession number 20030120282), vernier manipulators require an operator to closely and carefully visually inspect three different vernier scales (which can be-confusing, and which are often difficult to see clearly under a hood or on a cluttered laboratory workbench), write down various numbers (which can be accurate only to 1/10 of a millimeter, which is 100 microns), and then perform several mathematical calculations, to determine each and every location of interest (expressed in terms of three orthogonal distances from a similarly-recorded bregma location). These types of readings must often be taken multiple times during the course of a stereotaxic procedure.

A set of three orthogonal readings, in all three different axes with all taken or applicable at the same time, are referred to herein as orthogonal coordinates. It should be understood that location data points along all three axes are required to fully specify the three-dimensional location of an instrument tip, at a certain point in time.

Vernier manipulators are slow, tedious, and cumbersome to use, and they often lead to errors of measurement and/or calculation. Accordingly, the old vernier system was greatly improved and enhanced by the creation of digital manipulators.

As used herein, the phrase digital manipulator is limited in several ways. First, it is limited to “stereotaxic manipulators” as that term is conventionally understood and used by people who perform research involving small animals. As such, a manipulator does not need to be attached to a base plate or U-frame assembly; instead, it can be manufactured and sold separately, if it is designed to be affixed (such as by retrofitting) to a suitable device (such as a base plate with a U-frame and clamping devices) that can hold an animal stationary while a procedure is carried out.

The term “stereotaxic” formerly had several different meanings, but gradually, it has come to refer to a procedure in which location coordinates for an instrument tip or similar device are spelled out in a three-dimensional manner, using three numbers to represent the location and/or travel of some item at a particular point in time. In nearly all cases, the orthogonal system is used, with X, Y, and Z coordinates (also referred to as A/P, D/V, and M/L coordinates when an animal body is involved, as described in more detail below). However, in some cases, polar (spherical) or cylindrical coordinates (usually involving a position along the length of a central axis, coupled with a radius-type distance from the axis, and an angular measurement relative to a vertical or other reference plane) can alternately be used to specify stereotaxic coordinates.

As used herein, “stereotaxic manipulators” also is limited to devices of the type that are used in research involving animals. Such devices do not include or relate to instruments that are used in surgery on humans. Many large and highly expensive and sophisticated computerized instruments have been developed for use in human surgery; however, those typically cost multiple tens or even hundreds of thousands of dollars, and they are not suited for use for studying animals on a crowded laboratory benchtop. By contrast, the improved digital stereotaxic manipulators disclosed herein are carefully and specifically designed to have size and cost ranges that render them well-suited for research on animals, rather than for surgery on humans.

The reference to “digital” stereotaxic manipulators implies and requires that a stereotaxic manipulator must be designed to send electronic signals to a signal-processing device, in a manner that will allow the signal-processing device to display location coordinates for an instrument tip, during a stereotaxic procedure on a non-human animal. However, this does not require that a manipulator must contain the necessary signal-processing device or components, as part of the manipulator. Instead, a typical digital stereotaxic manipulator will simply have a multi-lead data cable (or a set of several two-lead wires) emerging from it, with a multi-lead connector at the end of the cable (or a two-lead connector at the end of each wire), designed to be plugged into a suitable card, slot, port, or similar interface on a computer or other electronic display device. Alternately, a digital stereotaxic manipulator might be provided without having a multi-lead data cable as a part of the system, and can be provided instead with three small connectors, positioned at or near each of the electronic reader heads; however, it should be noted that any such connectors should not be coupled directly to the reader heads, since that might create a risk of gradually dislodging or misaligning the reader heads, if cable connectors must repeatedly (over a span of months or years) be plugged onto or pulled off of the head connectors.

First Generation Digital Manipulations

The first stereotaxic holders and manipulators having digital readouts and “zeroing” capability were developed in the early or mid-1990's by a company called Cartesian Research (www.cartesianresearch.com), apparently working with substantial government support. These devices were a major advance over conventional vernier (manual) stereotaxic holders, which had been used for about 30 years with essentially no changes or improvements. However, those “first generation” stereotaxic holders apparently were never patented. The closest relevant patent located by the Applicants herein is U.S. Pat. No. 6,258,103 (Saracione 2001); however, its teachings and claims focus on other aspects of stereotaxic holders, and do not claim digitized signal, display, or zeroing systems.

Those digital stereotaxic holders, referred to herein as “first generation” stereotaxic holders, suffered from several limitations and drawbacks. They are large, bulky, and cumbersome, which means that they are difficult to place and use, on crowded benchtops in active research laboratories. They are also expensive, costing over $10,000 for each unit. In addition, no way was provided to replace an vernier manipulator, on an old stereotaxic holder having a perfectly good base plate and U-frame, with a retrofitted manipulator system digital measuring components.

To overcome the limitations of first-generation digital systems, and to provide less expensive, more convenient, and more easily usable systems, a company called myNeurolab, Inc. (St. Louis, Mo.; www.myNeurolab.com), a subsidiary of Coretech Holdings Inc., created “second-generation” digital stereotaxic holders, and began selling them in early 2002. These new devices were less bulky and more convenient (and therefore substantially easier to position and use on a crowded workbench) than the previous “first-generation” digital systems. They also were substantially less expensive than first-generation systems, and they provided manipulators that were specifically designed to be retrofitted, simply and easily, onto existing base plates and U-frame assemblies from vernier stereotaxic holders.

Second-Generation Digital Manupulations

“Second-generation” digital stereotaxic holders are described in detail in a separate copending patent application, which is owned by the same assignee herein (Coretech Holdings Inc., the parent company of myNeurolab, Inc.). That copending application, Ser. No. 10/036,231, has been posted on the official USPTO website, under accession number 20030120282. Its entire contents are incorporated herein by reference, as though fully set forth herein.

That application is copending with this continuation-in-part application, and it is not conceded to be prior art against this application. However, the most logical and straightforward way to understand the structural differences and advantages of the new “third-generation” digital manipulators, as disclosed herein, is by comparing them to “second-generation” manipulators. Therefore, a second-generation system is briefly described below, and it is illustrated in FIG. 2. Second-generation systems are described and illustrated in more detail, in utility application Ser. No. 10/036,231 (although they are not referred to by that phrase, in that application), which is available on the USPTO website under accession number 20030120282.

A “second generation” digital stereotaxic manipulator is illustrated in FIG. 2 herein, which is essentially identical to FIG. 2 in application Ser. No. 10/036,231, except for the addition of callout number 201 to specifically point out the turret base plate. As noted in application Ser. No. 10/036,231, components with callout numbers from 100 to 399 are the same components used on vernier (manual) systems, while components with callout numbers from 500 to 599 are the electronic reader heads and linear scales (and their mounting components) that interact to provide digital electronic signals that can be converted into digital location data along each of the orthogonal axes, for display on a separate display device or computer.

Digital manipulator 200 is mounted on a horizontal slide 180, which travels within a horizontal linear pathway established by a non-moving base 184. Base 184 can be screwed or bolted securely to one arm of a U-frame, including the same U-frame 104 that previously was used on an old vernier holder, as shown in FIG. 1. This allows a second-generation digital manipulator to be conveniently retrofitted onto an old vernier stereotaxic holder.

Control of the travel and positioning of slide 180 is provided by rotating knob 182, which is coupled to a threaded shaft 183 (shown in FIGS. 3 and 4) that is positioned below slide 180. Threaded shaft 183 rotates within a bushing 185 that is securely affixed in a non-moving, non-rotating manner within base 184. Accordingly, when threaded shaft 183 is rotated by an operator, using knob 182, the slide 180 and all components that are mounted above slide 180 (which includes all manipulator components with callout numbers of 202 and above) will travel along the Y axis (also called the A/P axis, as indicated in FIG. 1) in either an anterior direction (i.e., in the direction from the animal's tail, toward its nose), or a posterior direction (i.e., from the animal's nose, toward its tail).

It should be noted that stereotaxic manipulators sold by myNeurolab Company use “double-knob” structures, with a first large-diameter knob that can provide somewhat better control over rotation of the shaft (and travel of an instrument tip) during sensitive steps in a procedure, and with a second smaller-diameter knob (which can be rotated more rapidly) also provided at the end of the knob shaft, to enable rapid withdrawal of an instrument tip after the sensitive steps in a procedure have been completed.

The upper portions of manipulator assembly 200 are detachably mounted on one end of manipulator slide 180, under the control of clamping screw 204, which can either secure or release a circular turret base 202 (also called a “yoke”, due to its shape, as shown in FIG. 3). Turret base 202 sits on top of a base plate 201, which is securely screwed to the top of slide 180. The shaft of clamping screw 204 passes through a threaded hole in turret base 202. When tightened, screw 204 presses hard against a a non-rotating sleeve that extends upwardly from base plate 201 (comparable to rotating sleeve 3104, shown in FIG. 4). This prevents any inadvertent rotation of the turret base 202 and the manipulator, during use. If clamping screw 204 is loosened, the turret base 202 can be rotated around the sleeve, or it can be lifted off and removed, to allow the entire upper portion of manipulator assembly 200 to be detached from the sliding base 180, for purposes such as cleaning, replacement by a different manipulator assembly, etc.

As used herein, the base and slide components 180, 182, and 184 are regarded as part of manipulator system 200, since those base and slide components provide one of the means of “orthogonal control” (described below) over an electrode, blade, needle, or other instrument affixed to the horizontal manipulator arm 270. However, since the upper portions of the manipulator system 200 can be easily detached from the slide component 180 by using clamping screw 204 to release the turret base 202, some users do not regard the slide 180 as being part of the manipulator system 200. This is an arbitrary semantic distinction; so long as a reader recognizes that the manipulator slide 180 provides one of the three types or axes of orthogonal control over an instrument, it does not matter whether that-person considers manipulator slide 180 to be part of the manipulator assembly 200, or not.

As mentioned above, if clamping screw 204 is loosed, the manipulator assembly 200-DIG can be rotated in either direction about a vertical axis that is established by base plate 201 and turret base 202, while still sitting on top of manipulator slide 180. This allows the manipulator assembly 200 to be rotated until its upper portions (and an instrument, if one has been affixed to the V-block 290) are out of the way. This can be convenient during certain stages of a procedure, such as while an animal is being secured to or removed from the holder, and while an animal is being surgically prepared for a procedure.

However, when a second-generation system is used, any manual rotation of turret base 202, after a bregma reading has been taken, will render exact repositioning of the manipulator 200 impossible, despite the existence of etched alignment marks on turret base 202 and slide 180.

Therefore, in most stereotaxic holders, a squared coupling is provided on the top of turret disc 203. This squared coupling is provided by a raised shoulder on the top of turret disc 203 (illustrated in FIG. 4), which interacts with a square mount 207 (illustrated in FIG. 5) that is securely affixed to the bottom of vertical arm assembly 240. The bottom tip of vernier rod 248 (which is part of the vertical arm assembly 240, as shown in FIG. 5) extends roughly 1 to 2 cm below the square mount 207, which is pressed against (or which could be part of) end cap 251, which is part of the vertical arm 240. The lower tip of rod 248 fits snugly into an accommodating orifice in turret disc 203, and it is clamped into position by clamping screw 208.

If clamping screw 206 is loosened, the vertical arm assembly 240 can be lifted slightly, while the lower tip of vertical shaft 248 remains engaged with the orifice in turret disc 203, until the square mount 207 is lifted above the raised shoulder on the top portion of turret disc 203. After the square mount 207 rises above that shoulder, the vertical arm assembly can be rotated exactly 90 or 180 degrees, until it reaches a convenient “out of the way” position. The vertical arm assembly 240 is then lowered again and secured by clamping screw 208, so that various surgical, observational, or other steps can be performed while the manipulator arm or instrument remains in an out-of-the-way position. After those steps have been completed, clamping screw 208 can be loosened once again, and the vertical arm assembly can be lifted slightly, rotated once again exactly 90 or 180 degrees (i.e., back to its exact starting position), and then lowered into place and clamped tight once again, held in its proper position by the square coupling system.

Accordingly, the square-mount mechanism was developed and designed to allow a manipulator to be rotated temporarily out of position, if and when a need arises, and then returned to its exact starting position, in a manner that will not introduce substantial errors into any orthogonal measurements. That is a useful option; however, it does not allow any accurate orthogonal measurements to be made, while the manipulator is in a rotated position.

By contrast, third-generation digital systems as disclosed herein allow accurate orthogonal measurements to be calculated and displayed, even when the manipulator is in a rotated position. This provides a number of additional useful and previously unavailable options, when invasive procedures of various types are being carried out.

Turret disc 203 also can be rotated about a horizontal axle 205, which is held by the two yoke arms of the turret base 202. This rotation system uses a detente pin 221, which travels within detente sleeve 222, to provide a mechanism that enables the turret disc to be rotated into a true vertical position. When the turret disc 203 is pointed in a true vertical direction, the tip of detente pin 221 clicks into place in a drilled or machined depression 223, located in a circular groove 224 that is molded or machined into turret disc 203. The tip of detente pin 221 is pressed against turret disc 203 by spring 225, which is held under compression by sleeve cap 226.

If clamping screw 206 is loosened, the vertical arm assembly 240 can be rotated around horizontal axle 205. When this happens, the tip of detente pin 221 will be forced out of depression 223, but it will “ride” within circular groove 224. When the vertical arm assembly 240 is returned to a true vertical position, the tip of the detente pin 221 will click back into place in depression 223.

Although this system allows a second-generation system to be secured in a reliable and true vertical direction, any other angling of the manipulator system, using horizontal axis 205, will render the resulting measurements inaccurate and “pseudo-orthogonal”. This is a major limitation in second-generation systems, and it is addressed and corrected in third-generation systems as described below.

FIG. 2 has callout numbers that indicate the three linear scaling devices (502 for the A/P axis, 542 for the D/V axis, and 562 for the M/L axis) and their corresponding reader heads (514, 554, and 574, respectively). The electronic signals generated by those three reader heads can be used to generate digital position values at all times during a procedure. Those orthogonal values can be displayed in a clear and easily visible manner, on a computer monitor or other electronic display device, to indicate the exact location of an instrument tip at any given moment during a procedure, to an accuracy of a single micron (or even less, if more refined linear scales and reader heads are used). The display device preferably should provide for convenient “zero-ing” of the position values at any desired time, by an operator, to allow the displayed values to directly indicate all three orthogonal distances from a bregma or other “starting point” location, at any given moment during a procedure, without requiring any additional calculations by the operator.

The manipulator components located above the squared coupling base 207 can be regarded as forming three major subunits, or subassemblies, referred to herein as vertical arm 240, traveling block 260, and horizontal arm 270.

Vertical arm 240 allows control of the vertical positioning of an instrument tip, by rotating knob 241. When an animal is in a conventional position, with its feet resting on baseplate 102, movement in the vertical direction is referred to as either dorsal (i.e., upward, from the animal's feet toward its backbone) or ventral (i.e., downward). As illustrated in FIGS. 2 and 5, the vertical arm subassembly 240 comprises several distinct components. Threaded shaft 242, which is coupled directly to vertical control knob 241, is positioned between vernier rod 248 and stabilizer rod 249. Rods 248 and 249 are both securely coupled, at both ends, to vertical end caps 250 and 251. As vertical arm 240 is operated to move the traveling block 260 and the horizontal arm 270 up or down, two smooth-surfaced sleeves (also called bushings) slide along the smooth shafts of vernier rod 248 and stabilizer rod 249. These bushings, typically made of a hard plastic such as nylon, DELRIN™, etc., are mounted inside traveling block 260.

Vertical threaded shaft 242 is rotated under the control of knob 241, and it passes through an internally-threaded bushing 244, which is affixed in a non-rotaing manner inside traveling support block 260. Since bushing 244 cannot rotate, it is forced (and the entire traveling block 260 is forced) to travel in a vertical position, upward or downward, whenever threaded vertical rod 242 is rotated. In this manner, control over the vertical motion of traveling block 260 (and therefore of horizontal arm 270) is provided by control knob 241.

Horizontal arm 270 is similarly structured, so that rotation of knob 271 allows control of the medial and lateral positioning of the horizontal arm, and of an instrument and instrument tip. Conventionally, movement toward the left side of the animal is regarded and displayed as the negative direction, while movement toward the right side of the animal is regarded and displayed as the positive direction. This avoids a semantic problem, wherein any motion away from the central vertical plane of a animal (which usually passes vertically through the center of the spine, in vertebrates) would be regarded as lateral, while any motion that approaches the central vertical plane would be regarded as medial.

Horizontal arm 270 includes a threaded shaft 272, which rotates under the control of knob 271. Threaded shaft 272 is flanked by vernier rod 274 and stabilizer rod 276. An internally-threaded bushing 273, affixed in a non-rotating manner inside traveling block 260, causes the horizontal arm assembly 270 (and any instrument affixed to it) to travel in a medial or lateral direction when the knob 271 and the threaded shaft 272 are rotated. While the horizontal arm 270 travels, vernier rod 274 and stabilizer rod 276 both slide through bushings (or sleeves) with smooth internal surface, mounted inside traveling block 260.

At one end of horizontal arm assembly 270, located adjacent to knob 271, the vernier rod 274 and stabilizer rod 276 are securely affixed inside end cap 280. At the opposed end of horizontal arm assembly 270, end cap or “V-block” 290 is provided with a V-shaped notch, as shown in FIG. 2, with an internally threaded screw hole in its center. The V-shaped notch and the threaded screw hole in V-block 290 work together to allow any desired type of instrument to be temporarily yet securely affixed to the V-block 290.

As shown in FIG. 1, a typical instrument subassembly 300 that is adapted for use with stereotaxic holders includes a securing clamp 310, a vertical shaft 320, and an instrument head 330 at the lower end of the vertical shaft. A typical securing clamp includes: (i) a horizontal bar with an angled-wedge surface that fits into and accommodates the notch in V-block 290; (ii) a knob which rotates a threaded shaft that screws into the screw hole in V-block 290; and (iii) a rounded vertical clamp that fits around the vertical shaft 320, and which is provided with a wing nut or similar tightening screw that can be used to tighten or loosen the vertical clamp, so that the vertical shaft 320 can be adjusted to any desired “starting” or “baseline” height, and then secured at that height. After a procedure has commenced, the vertical shaft is not moved or adjusted by manipulating the vertical clamp; instead, the height of the vertical shaft (and therefore the height of the instrument tip) is adjusted, during a procedure, only under the control of the manipulator's vertical knob 241.

Any type of instrument (such as a blade, needle, electrode, etc.) that is desired for use in a particular type of procedure can be mounted to the lower end of the instrument shaft, using (if desired) a mounting structure referred to generically herein as “instrument head” 330.

Cartesian Axes, Orthogonal Measurements

Anyone who has used a stereotaxic holder, or who examines FIGS. 1 and 2, will recognize how the three adjustment knobs 182, 241, and 271 work together to provide three-dimensional control over the placement (positioning) and movement (travel) of an instrument tip, at any given moment during a test on an animal.

The manipulator slide 180, the vertical arm assembly 240, and the horizontal arm assembly 270 are positioned in an “orthogonal” arrangement; this means that each slide or shaft subassembly is perpendicular to both of the other two subassemblies. Using standard “Cartesian” coordinates (named after the French mathematician Rene DesCartes), they establish three “axes” of motion, which are conventionally designated as the X, Y, and Z axes, as shown in the lower left corner of FIG. 1.

Using the conventional directional terms used in neurology, the manipulator slide 180 and its knob 182 control any motion of the instrument tip along the “A/P” (anterior/posterior) axis. The vertical threaded shaft 242 and knob 241 control motion of the instrument tip along the “D/V” (dorsal/ventral) axis. The horizontal threaded shaft 272 and knob 271 control motion of the instrument tip along the “M/L” (medial/lateral) axis. These axis designations are shown by the arrows in the lower left corner of FIG. 1.

Many labs (and some instrument makers) also refer to these axes as the X, Y and Z axes, using the system that most students encounter in high school mathematics classes. When X, Y, and Z designations are used, the medial-lateral axis is deemed to be the X axis, the anterior-posterior axis is deemed to be the Y axis, and the dorsal-ventral axis is deemed to be the Z axis. These X, Y, and Z designations are also shown in FIG. 1, and in various items of prior art, such as U.S. Pat. No. 6,258,103 (Saracione, 2001).

As noted above, the type of stereotaxic holder which is illustrated in FIG. 1 is well-known prior art; similarly, all components shown in FIG. 2 which have callout numbers between 100 and 399 are prior art. Thousands of stereotaxic holders having this arrangement (or very similar arrangements) have been sold; they are standard equipment in nearly any neurology lab that works with surgical or other invasive procedures on small animals.

FIG. 2 illustrates scale-and-reader combinations that can be used to generate digital display data, along each of the three orthogonal axes. For measurements along the anterior/posterior (A/P) axis, a non-moving electronic reader head 514 is attached to the non-moving manipulator base component 184, by means of a mounting bracket. An etched linear scale 502 is secured to the moving slide component 180, also by means of a mounting bracket. As A/P control knob 182 is rotated, the horizontal slide 180, and the linear scale 502 which is attached to slide 180, will travel beneath the electronic reader head 514, thereby causing the electronic reader head 514 to generate and send electronic signals, via a data cable 599 (shown in FIG. 2) to a signal processing and data display unit, which will process the signals from reader head 514 to display the A/P value (position) of the instrument tip at any given instant during a procedure.

Similarly, a non-moving vertical dorsal/ventral (D/V) linear scale 542 is affixed to the vertical arm assembly 240, and an electronic reader head 554 is attached to traveling block 260. This allows vertical motion by traveling block 260 and reader head 554 to generate electronic signals that will pass through data cable 599, allowing the signal processing and data display unit to calculate and display the D/V value (position) of the instrument tip at any given instant during a procedure.

In addition, a horizontal medial/lateral (M/L) linear scale 562 is affixed to the movable horizontal arm 270, and M/L reader head 574 is affixed to traveling block 260. This allows M/L motion by horizontal arm 270 and M/L scale 562 to generate electronic signals from M/L reader head 574. These signals will pass through data cable 599, allowing the signal processing and data display unit to calculate and display the M/L value (position) of the instrument tip at any moment during a procedure.

Bregma Readings; Zero Points

During procedures on small animals, it is often necessary to establish the exact location of an electrode or other instrument tip, in or near the brain or skull. Since only minor variations in skull thickness and other anatomical structures occur between rats, positioning inside the brain is usually measured relative to a certain point called the “bregma”, which is visible on the top surface of the skull of a rat or mouse. As can clearly be seen by looking at a rat or mouse skull, the top surface is formed when several bone structures, usually called “plates”, fuse together to form a larger single structure. The remnants of the component plates remain visible, and are separated by shallow zig-zagging crevices between the plates. These crevices are usually called “sutures”, since they resemble stitches made of thread, or “fissures”, a term that refers to a furrow or crevice between two adjacent objects.

Since two anterior plates (left and right) merge with two posterior plates (left and right), two major fissure lines (called the sagittal suture, in the anterior-posterior direction, and the coronal suture, in the medial-lateral direction) cross and intersect with each other, in a generally “+” configuration.

This point of intersection, where the two major fissure lines cross each other, is called the bregma. It has a physical appearance similar to the “cross-hairs” used in rifle scopes, and in many types of camera viewfinders, microscopes, and telescopes. Its vertical position is established when the tip of an electrode or other instrument is lowered down onto the skull until the instrument tip barely touches the intersection of the two fissures.

Another important physiological location on the skull is called the “lambda”. This is another easily visible skull suture, which is posterior or “caudal” (i.e., closer to the tail) from the bregma. In many types of tests, the animal's head must be oriented in a “flat-skull” position, which indicates that the vertical height of the bregma and lambda locations must be the same. This can be accomplished by securing the animal in an approximately flat-skull orientation, and then making an initial measurement of the bregma and lamba heights. After the initial measurement indicates how far out of flat-skull alignment the skull is, a clamping screw that controls the height of the snout clamp is loosened slightly, the snout clamp is adjusted up or down, and the clamping screw is tightened again. Since the ear pins remain firmly in place during this adjustment, they provide an axis of rotation during this adjustment. One or two adjustments of the snout clamp height, by an experienced operator, are usually sufficient to provide a very close approximation of a flat-skull position.

As mentioned above, the bregma is regarded as a “zero point” location, in neurological tests on small animals. All other locations are described by indicating their distance and direction from the bregma, along each of the three orthogonal axes. Distances along all three axes must be indicated, to establish an exact location inside a brain. As an example, in the brain of a typical adult male rat weighing 250 grams, the center of the ventomedial nucleus of the hypothalamus would have coordinates of ML +0.5, DV −3.6, and AP −4.6 (all in millimeters). Three-dimensional maps or “atlases” of rat brains have been published, with enlarged photo-micrographs of the brains at various coordinates, and indicating the appearances of various structures within the brain. Such atlases can be located and downloaded via the Internet, from sites that can be located via search engines such as Google. One such site that was active when this application was written was http://java.usc.edu/cgi-bin/HBPReg/webdriver?MIval=index.html&tool=3DBrainAtlas.

As mentioned above, prior to 2002, nearly all stereotaxic holders generated orthogonal distance measurements that had to be measured and calculated manually, using “vernier” linear scales with distance marks (usually in millimeters) for each of the three orthogonal axes. Those measurements and calculations were cumbersome, awkward, and time-consuming, especially if a test was being carried out on a crowded workbench or under a hood, and they often caused errors in measurements. To create improved devices, a company called Cartesian Research (www.cartesianresearch.com) created “first generation” stereotaxic holders. Although those devices provided a major advance over stereotaxic holders having vernier scales, they suffered from several limitations and drawbacks, including their size and expense.

To overcome those problems and create a less expensive, more convenient, and more adaptable system, a company called myNeurolab, Inc. (a subsidiary of Coretech Holdings Inc., of St. Louis, Mo.; www.myNeurolab.com) created second-generation digital stereotaxic holders, and began selling them in early 2002. These new devices were less bulky and more convenient (and therefore substantially easier to place and use, on a corwded workbench), as well as substantially less expensive, than the older “first-generation” digital systems. They also used manipulators that were specifically designed to allow a new and completely digital manipulator to be retrofitted, simpy and easily, onto an existing base plate and U-frame assembly from a conventional stereotaxic holder.

This current patent application describes what are referred to herein as “third-generation” digital stereotaxic holders. These new systems have several-important improvements over first- and second-generation stereotaxic holders.

Accordingly, one object of this invention is to provide improved “third-generation” stereotaxic animal holders with digital readouts, having a “fine drive” manipulator control that can provide improved control over an instrument tip. This improved control can be 10, 20, 50, or even 100 times more precise than previously available control systems.

Another object of this invention is to provide improved digital stereotaxic animal holders that allow a manipulator and instrument to be rotated and/or angled in at least one and preferably two dimensions (or, stated in another manner, allowing a manipulator to be rotated and/or angled about at least one and preferably two axles), while retaining the ability to calculate corrected and adjusted orthogonal measurements that are highly accurate.

These and other objects of the invention will become more apparent through the following summary, drawings, and description of the preferred embodiments.

### SUMMARY OF THE INVENTION

A digital stereotaxic manipulator, for use in animal research, is provided with either or both of two enhancements. In one enhancement, at least one and preferably two rotary encoders are provided, to enable precise measurement of angular displacement of the vertical arm assembly (which supports the traveling block, horizontal arm assembly, and instrument) about a vertical axis and/or a horizontal axis. With assistance from software that applies mathematical adjustments to measured orthogonal coordinates, corrected and accurate orthogonal location coordinates can be calculated and displayed, even when the vertical and/or horizontal arms of the manipulator have been rotated and/or titled substantially. This enables various options and procedures that have not previously been available.

In the second enhancement, a “fine-drive” mechanism is added to the system, to provide an operator with more precise control over motion of an instrument. This can be done by mounting a radial gear to the main threaded vertical shaft in the vertical arm assembly, and providing a helical gear on a newly-added horizontal shaft. Rotation of the helical gear can be used to drive the radial gear, which will slowly rotate the main vertical shaft. If a 1:20 gearing ratio is provided by the fine-drive gears, this provides an operator with 20-times more precise control over vertical (dorsal-ventral) motion of an instrument. A spring-loaded detente mechanism is also provided, to enable the fine drive mechanism to pop out of position without damaging anything, if an operator attempts to rotate the main vertical control knob before disengaging the fine-drive mechanism.

If both enhancements are provided in a single manipulator assembly, the resulting manipulator is referred to herein as a “third-generation” digital manipulator, to distinguish these enhanced units from two generations of earlier models that do not have these enhancements but are otherwise very similar.

### BRIEF DESCRIPTION OF THE DRAWINGS

FIG. 1, which is prior art, discloses a conventional analog (or “vernier”) stereotaxic holder, with etched linear scales that must be inspected visually), for use with small animals such as rats. This figure illustrates a base plate, U-frame, ear pin, and snout clamp assembly, all of which can be retrofitted with a digitized second- or third-generation manipulator.

FIG. 2 illustrates a second-generation digital stereotaxic manipulator, as described in copending application Ser. No. 10/036,231. That system contains three electronic reader heads that are mounted next to adjacent linear scales, so that operation of any of the three slides or arms of the manipulator will cause the reader heads to send signals to an electronic display unit that will (i) display digitized position data for each of the three orthogonal axes, and (ii) provide convenient “zero-ing” capability, so that all coordinates can be set easily to zero values when an instrument tip is at a “baseline” point, such as the bregma location on a rat or mouse skull.

FIG. 3 is a perspective view of an assembled third-generation digitized stereotaxic manipulator, showing two rotary encoders that can be used to measure, to within about ⅙ of a degree, the position of a component that has been rotated about a vertical or horizontal axis. This view also shows the control knob and part of the shaft of a “fine drive” mechanism that provides greater precision when moving an instrument in a vertical (dorsal/ventral) direction.

FIG. 4 is an exploded view of the components that make up the lower half of a third-generation digital manipulator, showing the components that enable two rotary encoders to accurately measure angular displacement (partial rotation) of the manipulator about vertical and honzontal axes.

FIG. 5 is an exploded view of the components that make up the upper half of a third-generation digital manipulator, showing the components that provide a “fine-drive” mechanism that provides very precise control over rotation of the threaded shaft within the vertical arm of the manipulator.

FIG. 6 is a perspective view of the “fine-drive” mechanism, which illustrates: (1) a helical gear (also called a worm gear) that rotates under the control of its own knob; (2) a radial gear that is affixed to the main vertical threaded shaft of the manipulator, and that will rotate only once when the helical gear rotates multiple times; and, (3) a hinge-and-detente mechanism that allows the helical gear to disengage from the radial gear and move out of the way, without causing any damage to either gear, if an operator begins rotating the main knob on the vertical shaft while the fine drive mechanism is engaged.

FIG. 7 depicts a display unit that will display, on a touch-screen monitor, measured and calculated positioning data, at any given moment during a stereotaxic procedure on an animal. The measured values are generated by processing the signals from the three linear reader heads and the two rotary encoders. The calculated orthogonal coordinates are generated mathematically, to adjust the apparent or measured values from the three reader heads, and to generate corrected and adjusted values that account for any tilt and rotation of the manipulator.

### DETAILED DESCRIPTION

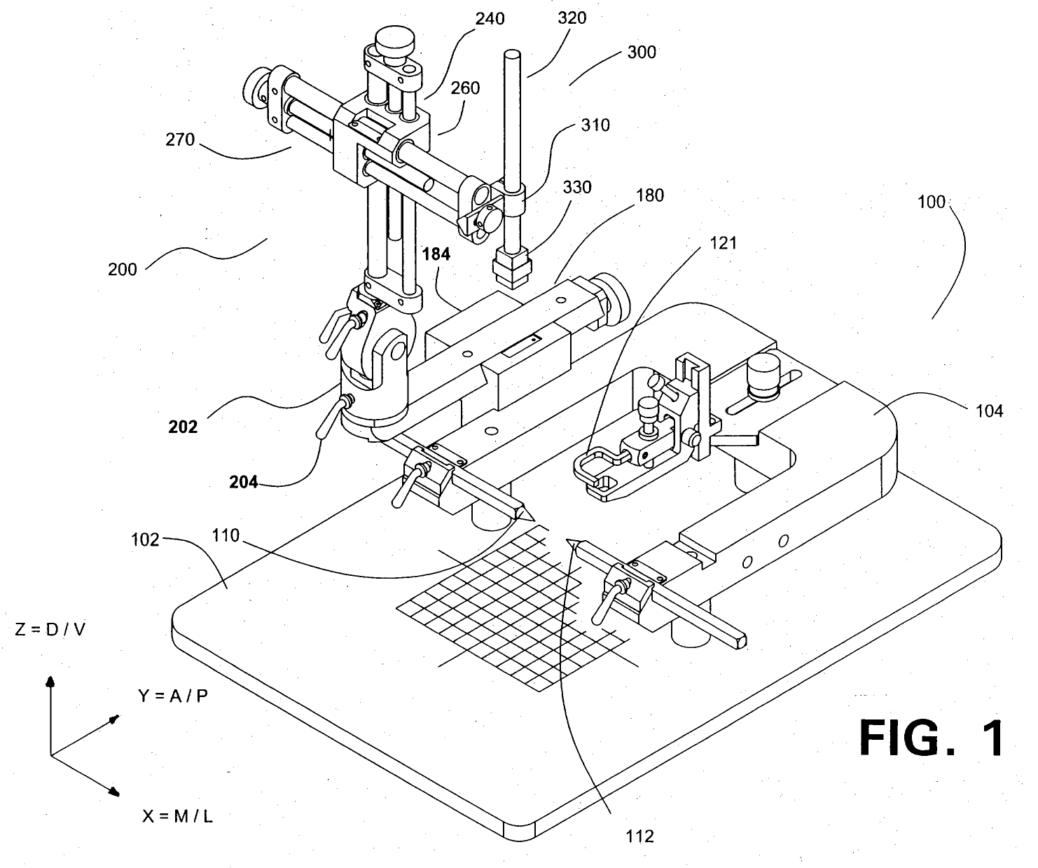

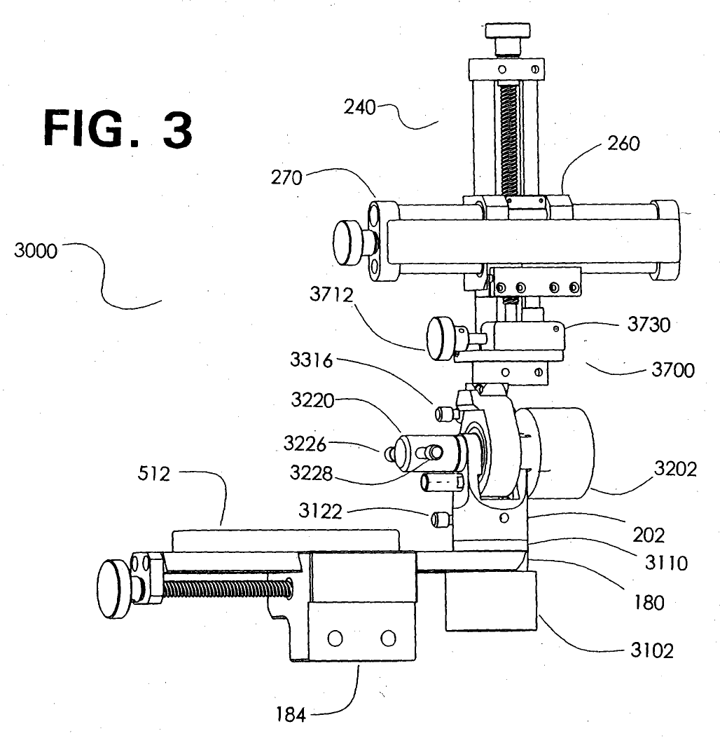

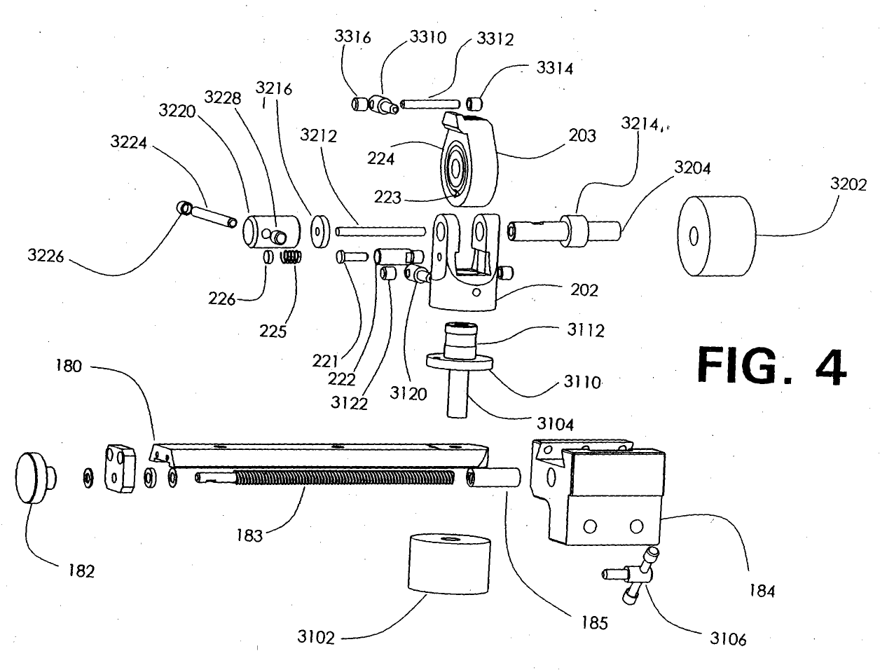

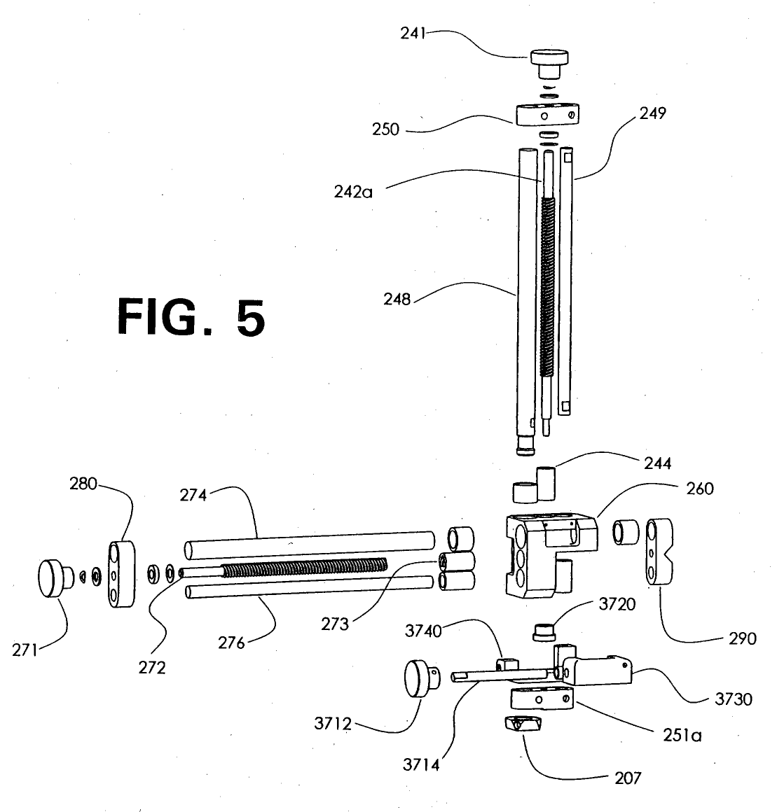

Referring to the drawings, FIG. 3 (assembled view), FIG. 4 (exploded view, lower half) and FIG. 5 (exploded view, upper half) depict a “third-generation” digital-readout manipulator system 3000, which can be retrofitted to a conventional stereotaxic holder base and U-frame, as shown in FIG. 1, that previously allowed only manual (vernier) readouts.

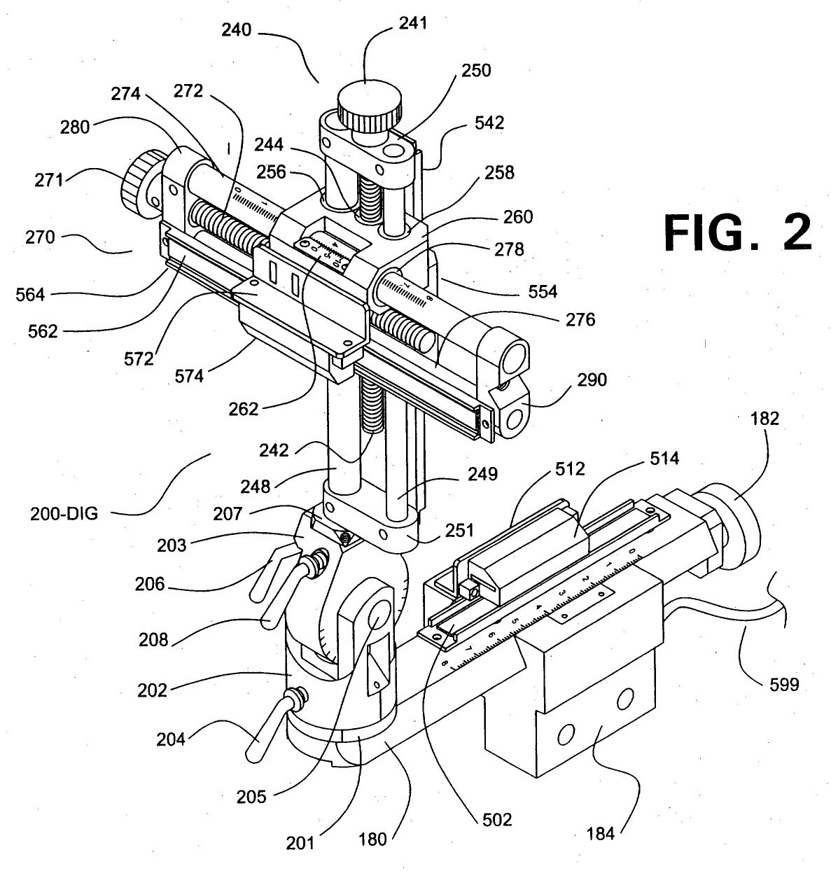

This third-generation digital manipulator uses electronic reader heads, to generate electronic signals that will be interpreted and displayed as digital orthogonal location data for an instrument tip, in the same manner as the 'second-generation” systems described above and in application Ser. No. 10/036,231, and illustrated in FIG. 2. Those reader heads, as shown in FIG. 2, are shown by callout numbers 514 (for the A/P axis), 554 (for the D/V axis), and 574 (for the M/L axis). Each reader head is mounted adjacent to a linear scaling devices, shown by callout numbers 502, 542, and 562, respectively, in a manner such that operation of the manipulator along any axis will generate relative motion between one of the reader heads and its linear scale.

In addition, as with the second-generation systems mentioned above, third-generation manipulator systems as disclosed herein can be retrofitted easily and conveniently to an existing stereotaxic holder having an vernier manipulator, simply by detaching the old vernier manipulator system 200 from U-frame 104 (in most cases, this can be done by simply unbolting the old manipulator base 184 from the U-frame 104), and replacing the entire vernier manipulator with a third-generation manipulator system 3000 as shown in FIG. 3.

However, four sets of newly-added components distinguish the third-generation system from the second generation system described previously. Those components include the following:

a. at least one and preferably two components, referred to herein as rotary encoders (often referred to commercially as “incremental encoders”), are provided, to allow precise measurement of the angular displacement (also called “partial rotation”) of one or more manipulator components around a vertical axis and/or a horizontal axis;

b. improved clamping/locking components are provided, to ensure that the manipulator arms and instrument tip will not waiver or drift when the manipulator is not being rotated;

c. a display device containing “programmable logic controller” (PLC) software is provided, which uses mathematical functions (including sine and cosine values) to enable accurately-adjusted orthogonal positioning data to be calculated at any instant for the instrument tip, despite any angular displacement(s) of the manipulator system; and,

d. a “fine-drive” mechanism is provided on at least one of the orthogonal drive screws, to enable improved control with greater precision over the motion of the instrument tip in at least one direction.

Each of these enhancements is described below, under its own subheading.

While components “a” through “c” as listed above interact with each other to provide additional and useful options for angular displacement, the “fine-drive” mechanism listed as component “d” can be added and used independently of the angular displacement mechanisms. Accordingly, various claims herein refer to either the angular displacement enhancement, or to the fine- drive enhancement, without necessarily coupling them together. However, in order to provide a state-of-the-art system that will enable and assist the best possible research, both the angular displacement components and the fine-drive mechanism should be provided, in a single coordinated system.

Accordingly, one of the features of this invention is that it discloses how to arrange and assemble a complete system that incorporates both of those enhancements, while also preserving all of the features and capabilities of second-generation digital systems as well. Therefore, a system that includes both sets of enhancements is referred to herein as a third-generation system, and a system that includes only one of those two enhancements would be regarded as a “2.x” generation system, which may offer an important enhancement over a second-generation system, but which falls short of a third-generation system.

It should also be understood that one of the goals of this invention is to provide a stereotaxic system that can determine and display orthogonal coordinates with a resolution of 5 microns or smaller (which is generally equal to half the diameter of most types of mammalian cells), and preferably with a resolution of 1 micron or smaller (since many neurons have fibrous extensions with roughly that diameter).

However, interactions with purchasers and users of second-generation digital systems have indicated that at least some of them do not want to have to work with accuracies less than 10 microns, which is 10 times finer than resolutions that can be achieved by using vernier systems. Indeed, a number of purchasers, after working for a week or two with second-generation digital systems, have specifically requested digital display panels that show resolutions of 10 microns, rather than 1 micron. Accordingly, certain claims below refer to display devices having resolutions of 10 microns or smaller, because that is the preference of some users, but it should be recognized that the systems disclosed herein will allow even finer resolutions, if desired.

Rotary Encoders; Angular Displacement

The manipulator base 184 and slide 180, and the A/P linear scale and electronic reader head that are mounted on slide 180, can be exactly the same, in second- and third-generation digital stereotaxic holders as described herein. Accordingly, a second-generation digital manipulator can be upgraded to a third-generation system, by adding several additional components to the second-generation system. The procedures and components that can be used for such an upgrading can also be used to conveniently describe the assembly and workings of the third generaton system.

To commence an upgrade, the clamping screw 204 of a second generation system is loosened and removed, thereby allowing the the turret base 202 and everything located above it to be disengaged from the horizontal slide 180. This will leave an open and unoccupied mounting hole near the end of slide 180.

Depending on the type of rotary encoder that is purchased, rotatable shaft 3104 can extend out of the rotary encoder, as part of the encoder, or it can be fabricated separately, and then inserted into an accommodating receptacle (or orifice, tunnel, etc.) in the rotary encoder. Because of certain fabrication and assembly requirements of the system disclosed herein, it was decided to fabricate shaft 3104 as part of a separate assembly (which also includes turret base plate 3110 and rotary sleeve 3112, as shown in FIG. 4), and insert the shaft into a rotary encoder 3102 that contains a shaft receptacle, rather than a protruding shaft. To prevent rotational slippage, the shaft 3104 and the encoder shaft receptacle both have machined flat surfaces, so that they are not perfectly circular.

Turret base plate 3110, which does not rotate, is screwed to the top surface of slide 180. Encoder shaft 3104 extends below it, and rotatable sleeve 3112 extends above. Both of those two components 3104 and 3112 are securely coupled or permanently affixed to each other, so that they rotate together with no slippage. The turret base 202 is then lowered onto the sleeve 3112 until it rests upon the base plate 3110, and the turret base 202 is then secured to rotatable sleeve 3112 by means of a clamping or locking screw 3120.

After the encoder 3102 has been secured to the bottom of slide 180, the shaft 3104 is lowered into the shaft receptacle in the encoder 3102, and base plate 3110 is screwed to the slide 180. In the next assembly step, turret base 202 is lowered onto the rotatable sleeve 3112, and secured to it by means of clamping or locking screw 3120, which uses a slidable lever that passes through the screw head (comparable to screw 3220, which is more clearly visible in FIG. 3) to generate torque on the locking screw 3120.

Any of several possible mechanisms can be used to lock the turret base in place and prevent any undesired rotation of the manipulator. In the mechanism illustrated in FIG. 4, a mounting brackets was used to enable a clamping or locking screw 3106 to be affixed to rotary encoder 3102, beneath slide 180. When clamping or locking screw 3106 is tightened, it prevents any rotation of shaft 3104 or encoder 3102.

In an alternate option, rotatable sleeve 3112 could be provided with a lower sleeve portion that cannot rotate relative to turret base plate 3110, and an upper sleeve portion that will rotate, relative to turret base plate 3110. If this type of two-part sleeve mechanism is used, a “set screw” (preferably of a type that can be tightened or loosened only by a screwdriver, hex wrench, or similar tool), positioned higher than locking screw 3120, can be used to lock the turret base 202 to the rotating upper portion of the sleeve 3112, while clamping or locking screw 3120 will have the tip of its shaft press against the lower non-rotating portion of the sleeve component 3112. In this manner, when clamping or locking screw 3120 is tightened, it will lock the turret base 202 to the lower, non-rotating part of the sleeve 3112, thereby preventing any undesired rotation of the turret base 202.

A second rotary encoder 3202 can be provided in the location shown in FIGS. 3 and 4, oriented with its shaft 3204 in a horizontal orientation. This will enable an alternate and/or additional type of angular displacement of the vertical and horizontal arms 240 and 270, and any instrument attached to the horizontal arm 270, around the axis that is established by horizontal encoder shaft 3204.

In the mechanism shown in FIG. 4, shaft sleeve 3214 is securely affixed to turret disc 203, by means of a set screw that presses into the indentation shown on the top of shaft sleeve 3214. This will lock the shaft sleeve 3214 to the turret disc 203, thereby ensuring that if the turret disc is rotated (this type of rotation is referred to herein as “tilting”, since it imparts an away-from-vertical tilting motion to the vertical arm 240 that is mounted on top of the turret disc 203), then the shaft sleeve 3214 will rotate along with the turret disc. The encoder shaft 3204 will also be securely locked to the shaft sleeve 3214, whenever the unit is assembled and in use, so that rotation of the shaft sleeve 3214 (under control of the turret disc 203) will also necessarily cause rotation of the encoder shaft, thereby causing encoder 3202 to generate an electronic signal that will accurately indicate the exact extent of the rotation. If an encoder is used that provides a centered shaft opening, rather than a projecting shaft, the encoder shaft 3204 can be machined from the same piece of metal that is used to make the shaft sleeve 3214.

The shaft sleeve 3214 is held, snugly but in a rotatable manner, within the aligned holes that pass through the two “yoke” arms of the turret base 202. It is held in place by screwing a threaded rod 3212 into an accommodating internally-threaded center tunnel in shaft sleeve 3214, and by emplacing a plastic washer 3216 and enlarged cylindrical screwhead 3220 on the other end of threaded rod 3212, and by tightening the enlarged screwhead 3220 until both the plastic washer 3216, and the enlarged “shoulder” portion of sleeve 3214, press snugly against the opposing yoke arms of the turret base 202 (as described below, adequate torque and tightness can be ensured by using a slidable lever 3224, which passes through screwhead 3220 and which is held in place by end caps 3226 and 3228).

If tilting of the manipulator is desired, the enlarged cylindrical screwhead 3220 is loosened. If no tilting is desired, or after a tilting operation has been completed and the system will be temporarily locked in place at a tilted angle, cylindrical screwhead 3220 is tightened, using sliding lever 3224 to generate a high level of torque. These types of cylindrical screwheads, which use sliding levers to generate high levels of torque (thereby ensuring that a clamping screw can effectively serve as a locking screw), are discussed in more detail below.

To distinguish between them, first encoder 3102 is referred to herein as a vertical encoder, since it has a vertical shaft and a vertical axis of rotation. Second encoder 3202 is referred to herein as a horizontal encoder, since it has a horizontal shaft and a horizontal axis of rotation. If desired, a stereotaxic holder and manipulator system can be provided with only a single such encoder, positioned in either a horizontal or vertical orientation, to create a “2.5-generation” digital system. This could add substantial value to the system, when compared to a first- or second-generation system that completely loses the ability to measure or calculate accurate orthogonal positions as soon as any angular displacement is introduced into a procedure. However, a full third-generation system, which provides two different encoders to allow precisely-measured angular displacements about two different axes, rather than just one, offers additional options and advantages, and justifies the relatively minor incremental expense of providing a second encoder.

Rotary encoders, such as encoders 3102 and 3202 as shown in FIGS. 3 and 4, are sold by companies such as IVO Gmbh. & Company (www.ivo.de), of Germany. In order to keep costs to a minimum, the types of rotary encoders that have been selected for use herein can be used for making precise angular measurements, but they are not adapted for also providing rotational power to the shaft. If desired, alternate types of encoders (including a class of devices usually referred to as “stepper motors”) could be selected and used, in which electrical energy is used to provide rotational power and motion to the shaft.

Each of the encoders 3102 and 3202 shown in FIGS. 3 and 4 comprises: (i) a main cylindrical body, which can be affixed to a larger device (such as the bottom of manipulator slide 180) by using mounting brackets or holes that are normally provided as part of the main body housing; (ii) either a rotatable shaft that emerges from the main body as a part of the unit that is sold, or an open shaft receptacle that can be used to insert a shaft, thereby allowing the purchaser of the encoder to provide and use an external shaft component with specialized adapters, connecting attachments, or other components; and, (iii) a data cable or connector, which will allow electronic signals to be sent to an external unit that can interpret and process the electronic signals, and display or otherwise utilize the data that is carried by the signals.

When an encoder shaft is rotated, the encoder will send out signals, through a data cable, which correspond to the exact amount of rotation that occurred. When a stereotaxic holder is involved, the power that drives the rotation normally will be provided by an operator or technician, who will manually adjust the angle or tilt of the manipulator. This is normally done by loosening a locking screw, rotating the vertical and/or horizontal arms about a vertical and/or horizontal axis and adjusting it until it reaches the desired tilt (with the help of the display panel, if desired), and then tightening the locking screw again, to hold the angled or titled arm(s) securely in their new position.

This type of adjustment, by an operator, will provide a controlled amount of “angular displacement”, a term used interchangeably herein with “partial rotation”. These terms indicate that when an encoder is used in a stereotaxic manipulator, the shaft will not be spinning through multiple revolutions. Instead, the shaft will travel only through a limited arc that is only a fraction of a single revolution.

A reasonably inexpensive type of rotary encoder that can divide each revolution (i.e., 360 degrees) into 2000 increments has been selected for use herein, because it is regarded as sufficiently accurate for most uses; however, more precise encoders are also available, at greater expense, if desired. Since 2000 increments per revolution, divided by 360 degrees per revolution, is equal to 5.556 increments per degree, an encode that can divide each revolution into 2000 increments can provide an accuracy equal to 1/5.556 degrees, which is equal to 10.8 minutes of arc (in the standard “English” system for measuring arc, each degree is divided into 60 minutes, and each minute is divided into 60 seconds). For comparison, it should be noted that the visible marks that are etched into a typical turret base (which corresponds to the vertical axis of encoder 3102) have 5 degree increments, and the visible marks etched into a typical turret disc (which corresponds to the horizontal axis 3202) have 2 degree increments. Therefore, rotary encoders can provide roughly 30 times greater accuracy than manual readings, when turret base 202 is rotated about a vertical axle, and they can provide roughly 10 times greater accuracy than manual readings, when turret disc 203 is rotated about a horizontal axle.

When one considers the various options for precise, controlled, and digitally-recorded angular displacements that can be provided by one or preferably two of these rotary encoders, one can recognize how these devices can be used to provide a wide range of different “angles of approach”, for an instrument tip that must be invasively inserted into the brain or spinal cord of a rat or other animal until the instrument tip reaches a precise targeted location within the brain or spinal cord. Among other advantages, the use of various different “angles of approach” can allow an operator to avoid and spare especialy sensitive, vulnerable, and important portions and regions of the brain and brainstem, when conducting an invasive neurological injection, electrode implantation, or other procedure.

If desired, a rotary encoder and/or stepper motor can also or alternately be mounted above instrument shaft 320 (shown in FIG. 1), to allow controlled and/or accurately measured rotation of an instrument and/or instrument tip.

When rotary encoders are used to enable precise measurements of angular displacements in stereotaxic holders, it is generally preferable to use certain precautionary measures, to reduce the risk that such displacements might introduce small but potentially troublesome inaccuracies in any resulting measurements. Two such precautionary measures deserve mention in particular.

First, the number of such angular changes should be limited, and when possible, they should be carried out before a procedure begins, and before a bregma location is established and used as a “zero point” for subsequent measurements during the procedure. Second, after an angular displacement has been completed, the turret base 3150 and the turret disc 3170 should both be tightly locked in place.

The second precaution is important, and has led to a design change, because the Inventors' experiences in developing and testing these systems indicated that conventional angled clamping screws, as used in vernier manipulators or in second-generation digital systems (such as clamping screws 204, 206, and 208, as shown in FIG. 2), were not adequate for this task. Therefore, a different type of locking mechanism was selected and adapted to this use, as described below.

Improved Clamping Screws for Angular Locking

The angular locking mechanisms that were chosen for use in third-generation digital systems as disclosed herein merit attention, because the requirement of truly secure and reliable angular locking has led to an additional enhancement of these devices. Rather than using conventional angled clamping screws, such as clamping screws 204, 206, and 208 as shown in FIGS. 1 and 2, a stronger and more secure (and therefore preferable) locking system preferably should be provided, by using an alternate type of clamping screw that can fairly and accurately be referred to as a locking screw.

In this improved mechanism, an enlarged cylindrical screwhead (such as locking screwhead 3310, shown in FIG. 4) is coupled to a threaded shaft. A slidable lever 3312, which has substantial length and which can be used to generate a large amount of torque on screwhead 3310, is passed through a smooth-surfaced tunnel or orifice that passes through screwhead 3310. End caps 3314 and 3316 are secured to both ends of lever 3312, allowing lever 3312 to slide freely within the tunnel that passes through screwhead 3310. At any given moment during a stereotaxic procedure, lever 3312 can be slid in either direction, to enable the operator to generate high levels of torque on the locking screw 3310. This can ensure a very tight and secure locking operation.

Three sliding-lever locking screws are provided in third-generation manipulator 3000. Those three locking screws are most easily visible in the exploded view of FIG. 4, and include cylindrical screwheads 3106 (used to prevent any rotation of the turret base around the vertical axis established by encoder shaft 3104 and sleeve 3112) and 3220 (used to prevent any rotation of the turret base around the horizontal axis established by encoder shaft 3204).

If a normal second-generation design were used, the third locking screwhead 3310 would not require high torque, since it merely secures the square mounting assembly (in particular, the tip of the threaded screw shaft that is attached to screwhead 3310 presses against the lower tip of the vertical vernier rod 248, which is part of the vertical arm assembly 240, and the lower tip of vertical vernier rod 248 rests snugly inside an accommodating tunnel that is drilled or machined into the turret disc 203).

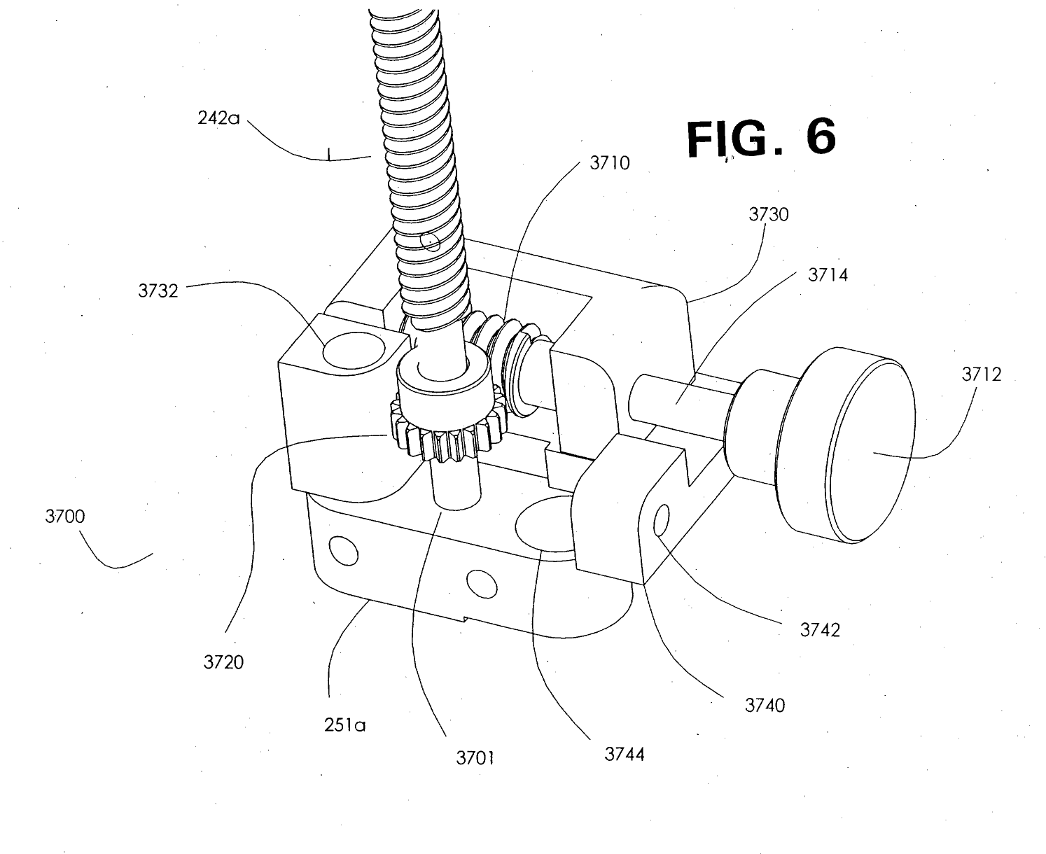

However, in the third-generation system disclosed herein, a “fine-drive” system (described below) uses a horizontal helical gear 3710 (sometimes called a “worm gear” in the vernacular), shown in FIG. 6, to enable finely-controlled rotation of the vertical threaded shaft 242a. This system uses a spring-loaded detente mechanism to enable the helical gear 3710 to be automatically pushed out of place, if it is still engaged when an operator tries to rotate the main vertical control knob 241. Because those mechanisms can exert force and torque against various components of the vertical arm structure, and can eventually lead to undesired “drift” out of proper alignment (especially if used repeatedly, over the course of numerous procedures), a high-torque locking screw with a sliding lever mechanism is preferred for use in that location. If desired, the lower tip of vertical vernier rod 248 can be provided with a machined flattened surface, to ensure against such gradual drift.

Computerized Calculations of Angular-Offset instrument Positions

When a stereotaxic manipulator is being used to control an instrument tip that must be measured accurately at a level of microns, the tasks that arise when calculating and displaying true and accurate orthogonal data, for location and travel of the instrument tip during a procedure, will be directly and unavoidably affected by the introduction of angular displacements in the manipulator system. Indeed, the very nature of an angular displacement is that it will take the system out of, and away from, the orthogonal arrangement and alignment that is normally used and depended upon, during a stereotaxic procedure. This will convert any apparent and unadjusted orthogonal measurements, coming directly from the three linear reader heads, into what are referred to herein as “pseudo-orthogonal” measurements.

However, angular displacements can be accounted for, and factored into the system, by the use of various mathematical calculations that involve trigonometric functions (such as sine and cosine values) that can be calculated accurately, if an angular displacement is known.

Since these types of calculations are ideally suited for computers, the assignee company that owns this patent application has arranged for a group of outside contractors, who are skilled in the art of writing software for devices that are called “programmable logic controllers” (PLC's), to prepare an embedded software program that will:

(i) display the horizontal and vertical angular displacements that have been provided by the two rotary encoders, as well as the measurements that are generated by the three electronic reader heads, at any given moment during a procedure; and,

(ii) use trigonometric values (which will depend on the angular displacements, at any given moment during a procedure) to adjust and correct the “pseudo-orthogonal” values that are being measured by the electronic reader heads at that moment, and convert those “pseudo-orthogonal” values into adjusted and accurate true orthogonal positioning data, which can be conveniently displayed on a suitable display device at any given moment during a procedure.

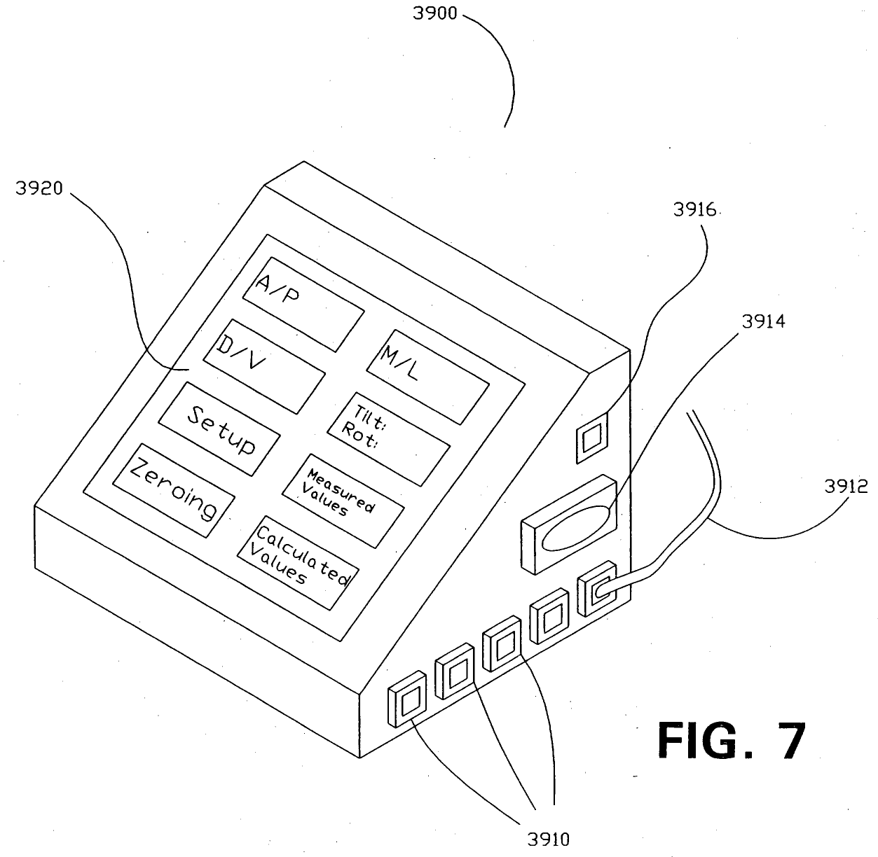

FIG. 7 depicts a stand-alone display device 3900 that has been programmed with software to render it suitable for interacting with a third-generation digital manipulator as disclosed herein. Suitable display devices, containing all of the necessary connectors for electronic data cables, and containing programmable integrated circuits, memory storage devices, and a display screen that also allows commands to be entered into the device by touching the screen, are sold (without specialized software) by companies such as Siemens (www.siemens.com).

Alternate means are also available for allowing a human operator to enter commands into an electronic system, without requiring complete alphanemeric or other keyboards. Examples of such means are provided by various designs for remote control units used for televisions and other electronic systems, and by the types of hand-held devices that are used for playing video games.

The display device 3900 in FIG. 7 is shown as having five small connectors 3910, to accomodate five different two-lead or four-lead data cables, such as cable 3912 (from the three linear reader heads, and the two rotary encoders). These types of connectors can be comparable to conventional telephone-type connectors which use plastic “click-type” fittings to ensure a good connection. Alternately, a single multi-lead connector (comparable to a serial, parallel, or joystick-type connector on a conventional desktop computer) can be provided, to interact with a “wiring harness” assembly that will branch out into separate wires that can be connected to the distributed reader and encoder locations on the manipulator. Normally, such connectors should be on the back of the device, rather than the side; they are shown on the side panel of display device 3900 merely for illustrative purposes.

If desired, an infrared sensor 3914 can be provided, to enable touch-free activation (such as by motion of a hand in front of the sensor) of a preprogrammed function. For example, this can enable the operator to activate, any number of times during a procedure, a routine that will record and store all readings and calculated orthogonal coordinates, along with a precise time each such recording is made, each time the infrared sensor 3914 is triggered.

Input port 3916 is also shown on the side of display device 3900. This port can be a USB, Firewire, or similar input port that is designed to enable the display device to be connected to a conventional desktop or laptop computer. This can enable two important functions. First, it can enable any operator to load software (include updated and enhanced versions of software that will become available in the future) into the programmable circuits and chips that are contained inside display device 3900. Second, it can enable the display device 3900 to continuously send data to a computer, during a procedure on an animal. This will allow the data that are being gathered, during a procedure, to be processed, stored, displayed, or otherwise handled, in any way that the computer is programmed to carry out. Accordingly, this type of connection, between a display device and a computer, can enable a wide variety of additional useful functions to be developed for these enhanced digital manipulators, after they become common in research labs.

If desired, an array of several different display panels can be provided on the face of the display device. However, to provide maximum flexibility and utility (and to enable additional enhancements as the software is developed and refined, in the future), a single relatively large display panel, which can be divided into various different functional regions at any given time, under the control of an operator, and which can also enable “touch-screen” functions, is preferred for use herein. By way of example, the display boxes shown in FIG. 7 can provide a “main menu” or “default” display. If the operator touches or presses the rectangle marked “Setup”, a different display will be activated, to allow the operator to set a clock or timer mechanism and/or conduct various initialization functions and other activities. When the “zeroing” button is pressed, a separate warning display should be activated, which will give the operator a chance to either complete the zeroing procedure if he or she wishes to do so at that moment, or to cancel the command and return to the previous readings, if the zeroing button was activated inadvertently.

In the default screen, the A/P (anterior/posterior), M/L (medial/lateral), and D/V (dorsal ventral) panels can be programmed to normally display calculated (i.e., adjusted) values, which will account for any rotation and/or tilting of the two rotary encoders. However, for checking and confirmation purposes, the operator can press other command buttons that will cause the display to toggle back and forth between (i) uncorrected and unadjusted “measured values” that are being emitted by each reader head, and (ii) calculated and adjusted values that take into account the data from the rotary encoders.

All of these functions (and various others as well) can be programmed into commercially available PLC devices, by people skilled in the art of writing PLC programming software.

If desired, a display device also can be provided with a small printing device (either internally, or via a connector cable) that will allow it to print out location coordinates, times, and any other data on a strip of paper. Such printers can use thermal printing on heat-sensitive paper, if desired, to eliminate any need for ribbons, ink, toner, etc.

When suitable software has been loaded into the programmable integrated circuits in a suitable display device, the programmed device will have all of the components and software necessary to carry out each and all of the following steps:

(1) receiving electronic signals from a third-generation digital manipulator having three linear reader heads and two rotary encoders;

(2) processing each and all of those signals, to convert them into both measured (i.e., unadjusted) and calculated (i.e., rotationally adjusted) orthogonal values;

(3) display all measured and/or calculated orthogonal values, at any point in time during a procedure;

(4) set all measured and/or calculated orthogonal values to zero, under the control of an operator, at any desired moment during a procedure, such as when an instrument tip reaches a bregma location or other “zero-coordinate” location; and,

(5) display subsequent values as orthogonal distances relative to the bregma location or other location that was established as a “zero-coordinate location” by the operator.

The assignee company has been assured, by the contract programmers who are writing the code for the PLC devices, that such calculations are entirely feasible and reasonable, and can be written into a suitable software program that can be loaded into a commercially available PLC device that can carry out all of the functions listed above. Because of the proprietary nature of these systems, and to reduce the risk of unauthorized copying and use, this type of software program is usually preloaded into a small electronic device that is designed to be plugged temporarily into only one or a limited number of models of PLC devices, and a password or serial number is usually required to transfer the software from the encoding device, into the memory circuits of the PLC device (which typically use circuitry that falls into the “EPROM” (electronically programmable read-only memory) or similar classifications, allowing the software to be upgraded if necessary, but only by authorized installers or users).

As of the date this application is being filed, initial functionality presentations have been made, but the software code has not been completed or finalized, and it has not been provided to the inventors or the assignee company by the contract programmers. The inventors, who do not specialize in writing computer code (and who do not have even an ordinary level of skill, in writing software for PLC devices), do not know the details of how the programming code is being written, or what it says. Instead, the inventors assert and declare that they have been informed, by contract programmers who write software for PLC devices, that such code can indeed be written by those skilled in that particular line of art, after the design, layout, and dimensions of a third-generation digitized stereotaxic manipulator have been adequately explained, and after a complete prototype of a third-generation manipulator (containing three orthogonal reader heads and two rotary encoders, along with the manufacturers' information that accompanies those devices) has been provided to the software developers, for testing and debugging purposes.

Alternately or additionally, it is also possible to create similar software for a desktop or laptop computer that runs a widely-used operating system, such as a recent version of Microsoft Windows and/or Apple Macintosh, or for various other types of computers that may be running a platform language such as C, Unix, Fortran, etc. If desired, such computers can use an external interface device, which can be designed to plug into a universal serial bus (USB), Firewire, or other input port on the computer. Alternately, a suitable interface between the digital manipulator and the computer can be provided by an internal card that can be inserted into a computer's mainboard, by means of a PCI or other widely used type of expansion slot.

The Fine-Drive Mechanism

FIGS. 3, 5, and 6 illustrate the components of a “fine drive” system 3700, which can be used to provide extremely precise control over the rotation of the vertical threaded shaft 242. This allows extremely precise control over the travel and positioning of the instrument tip, during any critical steps during a procedure.

Since threaded shaft 242 will have to be modified slightly, compared to a conventional shaft in a second-generation system, to provide the fine-drive option in a third-generation system, the modified threaded shaft is referred to in FIG. 6 by callout number 242a. The modifications required to shaft 242a involve: (i) making it slightly longer, so that it will extend down to the fine drive mechanism 3700; and, (ii) providing a non-circular shaft segment 3701 near the bottom end of shaft 242a (such as by providing a flat facet on one side of the shaft, which can interact with a set screw or an accommodating non-circular orifice through the radial gear 3720), so that radial gear 3720 can be securely affixed to the non-circular shaft segment 3701 in a manner that will not allow any rotational slippage between shaft 242a and radial gear 3720.

The main components of the fine-drive system include a helical gear 3710 (also called a worm gear, as noted above), which is rotated under the control of its own control knob 3712, a radial gear (also called a star gear) 3720, which is affixed securely to vertical threaded shaft 242a, a housing component 3730 which rests on a base plate 3740, and a spring-loaded detente mechanism which is screwed into hole 3742 of the housing.

Helical gear 3710 is provided by one or more thread-like ridges that are wrapped around a shaft in a helical manner. In order to provide secure and reliable engagement of this helical gear with radial gear 3720, the helical ridges preferably should be oversized and/or blunt-crested, as distinct from the closely-spaced and sharp-crested threads found on conventional machine screws. Accordingly, when a cylindrical shaft has that type of large, blunt-edged helical crest wrapped around it, it roughly resembles a worm that is-wrapped around a tube (hence the common name, “worm gear”). If desired, these types of helical gears can also be referred to as worm shafts, worm drives, or worms. They are sold commercially, by companies such as Stock Drive Products (www.stp-si.com), along with shafts that will fit snugly within the worm gears. Typically, the drive shaft and the orifice that passes through the worm gear will have accommodating flat sides (so that the shaft and the orifice both have a shape that roughly resembles the letter “D”), to prevent rotational slippage. A set screw, positioned and sized so that it will not interfere with operation of the gears, is normally used to prevent longitudinal slippage; alternately, a helical gear can be bead-welded at one end, or otherwise attached to its shaft.

The ridges on helical gear 3710 engage a conventional circular gear, referred to herein as radial gear 3720 (based on its general shape, with its cogs extending outwardly in a radial direction), to clearly distinguish it from helical gear 3710. The extended ridges or cogs on radial gear 3720 generally should be angled in a manner that matches and interacts with the slope of the ridges on helical gear 3710, so that as the helical gear 3710 is rotated, normally under the control of a human operator who is manipulating knob 3712 (or possibly under the control of a drive mechanism that is under the control of a computer or other programmable device), the apparent travel of the helical ridges will cause the radial gear 3720 to rotate slowly.

As can be understood by examining the figures, the value of providing a geared mechanism of this type resides in the following fact: by adjusting the spacing and slope of the helical ridges on the helical gear 3710, and the diameter and the number of cogs on the radial gear 3720, it is possible to provide gearing combinations that allow multiple complete revolutions of the helical gear 3710 to drive a single complete revolution of the radial gear 3720. Gearing ratios that range from about 1:8, up to about 1:80, are fairly common, and even wider ratios can be provided if desired.

Therefore, a gearing ratio can be provided that will allow much finer control, over the rotation of vertical shaft 242a, than can be achieved without this mechanism. For example, if a gearing ratio of 1:20 is used (which has been found to be suitable and useful for use as disclosed herein), if the helical knob 3712 is rotated through some fixed amount of arc (such as 90 degrees), vertical shaft 242a will be rotated only 1/20 of that amount (i.e., only 4.5 degrees). Therefore, this mechanism provides better, finer, and more precise control over travel of an instrument tip in the vertical (dorsal/ventral) direction.

During any invasive procedure, a skilled operator will be (and indeed, should be) paying much closer attention to the animal, the instruments, and the procedure, than to the manipulator system that he or she is using. Therefore, nearly any operator will occasionally (or even frequently) forget to disengage the fine-drive mechanism from the vertical shaft, before attempting to use the main vertical knob 241 to rotate vertical shaft 242a. Therefore, a disengagement mechanism that prevents damage to either of the helical or radial gears preferably should be provided, if a fine-drive mechanism is included as a component in a stereotaxic manipulator.

This type of automatic disengagement mechanism is provided in the system shown in FIG. 6, as follows. The housing component 3730 rests on top of a base plate 3740 (if desired, both of those components can be machined from a single piece of metal, to provide a single housing structure). Base plate 3740 rests on top of modified end plate 251a, which is part of the vertical arm assembly 240. Modified end plate 251a is essentially the same as corresponding end plate 251 as shown in FIG. 2, except that a centered orifice has been drilled through it, to accommodate the lower end of the elongated threaded rod 242a.

Housing component 3730 and base plate 3740 are able to rotate about a vertical axle that is provided by stabilizer rod 249, which passes through orifice 3732, which passes through housing component 3730 and/or base plate 3740. When the fine drive mechanism is in use, housing component 3730 is held in position (so that helical gear 3710 engages radial gear 3720) by means of a spring-loaded detente system, which takes advantage of the placement of the vertical vernier rod 248, which passes through orifice 3744 (shown in FIG. 6) in end plate 251a. A cylindrical ball plunger having an external threaded housing (such devices are sold by companies such as McMaster-Carr, www.mcmaster.com) is screwed into threaded orifice 3742, to a depth that causes the spring-loaded ball plunger to momentarily press against vernier rod 248 as the system is pushed into an engaged position, when the operator wishes to engage and use the fine drive system.

Subsequently, when the operator is finished using the fine drive system, he or she can simply swing the fine drive system out of the way, simply by exerting lateral force on fine drive knob 3712. Alternately, if the operator forgets that the fine drive system is still engaged, and attempts to rotate the main vertical knob 241, the force that is exerted on the angled surfaces of the helical gear 3710, by the angled surfaces of the cogs on radial gear 3720, will exert a pushing force on the shaft 3714 that will be sufficient to depress the detente mechanism (i.e., the spring-loaded ball plunger), and cause the fine drive system to swing out of the way, with no damage or inconvenience.

Example of Usage During Enhanced Cerebral Injections

The newly-provided functionality provided by the third generation digital stereotaxic manipulator described herein can be highly useful and even crucially important, in numerous types of neurological procedures, for a number of reasons that will be recognzied and appreciated by those skilled in the art. Among other advantages, it will enable researchers to use new and alternative approach routes, in invasive procedures that involve injection of various types of compounds into a brain of a rat or mouse. Such compounds can include, for example, experimental drugs, neuroactive polypeptides, genetic engineering vectors, genetically engineered cells, and stem cells.

It also should be noted that a recently developed and potentially important type of instrument tip can be used in these types of invasive procedures, to provide still greater flexibility and options for injecting extremely small quantities of compounds into brain or spinal tissue. This system is described in U.S. Pat. No. 5,792,110 (Cunningham, 1998), which is licensed to the same assignee company herein. Briefly, this injector system uses a thin yet relatively stiff cannula to approach a certain region of brain tissue. When the tip of the relatively stiff cannula reaches a desired location, an extremely fine hollow needle energes from it, at a controlled angle, and to a controlled distance. When the needle tip is fully extended out from the instrument tip, a single cell or a few cells, or a very small quantity of a drug solution or suspension, can be squeezed out of the needle tip, using a micropipetting device. After a very small quantity of material has emerged from the needle tip, the needle tip is retracted slightly, such as to a ⅔- or ¾-extended position, and a small additional quantity of liquid is squeezed out. The needle tip is then retracted a bit more, such as to a ⅓ or ½ position, and a small additional volume of liquid is squeezed out. The needle tip is then fully retracted, and the instrument tip is rotated by a controlled amount (such as 90 or 120 degrees), in a manner that does not cause any addition intrusion into or damage to the surrounding tissue. The needle tip is then extended as far as possible in the new direction, and a very small quantity of liquid is ejected; the needle tip is partially retracted, a small additional quantity of liquid is ejected, etc.

In this manner, multiple injection sites that are located in a cone-shaped array can be established, with minimal tissue damage or disruption.

Just as importantly, very small numbers of cells (or very small quantities of any experimental drug of other compound) can be released and deposited, at each injection site within that array. This is highly valuable for injecting cells or compounds into brain or spinal tissue, because of a certain type of cellular and physiological reaction that occurs within CNS tissue that is protected by the blood-brain barrier (BBB). Because antibodies cannot penetrate through the tight-junction capillary walls that make up the BBB, in the manner that occurs in a normal immune response of the type that occurs throughout the rest of the body, BBB-protected CNS tissue evolved a different mechanism for protecting itself against apparent intruders. This mechanism involves a “walling off” response, in which CNS cells (mainly glial cells, which cannot receive or transmit nerve signals, and which instead serve as helpers and supporters for neurons inside CNS tissue) form tight and essentially impermeable clusters or clumps of cells, surrounding apparent invaders. Tightly-packed clusters of glial cells that are forming an isolating and defensive capsule can prevent nutrients from reaching (and thereby feeding and supporting) encapsulated bacteria or viruses, and they can also greatly reduce the ability of bacterial toxins or infective virus particles to escape from the defensive encapsulation, and attack other cells in the CNS.

That type of defensive encapsulation response is highly useful and effective, in defending against viral or invaders. However, it creates a major hindrance that will block and drastically reduce the potential benefits (or research-related effects) that could otherwise be received from an injection of a compound into BBB-protected brain or spinal tissue.

In general, the likelihood of a defensive encapsulation response is believed to increase in a manner that is proportional (and possibly exponentially related) to the volume of a foreign compound or carrier liquid that is injected into a particular injection site, in brain or spinal tissue. In layman's terms, this is analogous to a small animal (such as a mouse, rat, or squirrel) being able to enter a house, without triggering an alarm system that would easily detect entry of a human. Therefore, an encapsulation response, which would render an injection of a desired compound useless and ineffective (or at least less useful, and less effective), will be minimized, and in many cases avoided entirely, if only extremely small quantities of a foreign compound or cell preparation are injected into a particular injection site.

However, for obvious reasons, the ability of an experimental drug, genetic engineering vector, or suspension of genetically engineered cells to exert a desirable or research-enabling effect inside CNS tissue will also depend heavily on the quantity of the drug, or the number of ccopies of the vector or cells, being injected.

Therefore, the injection system described above, using a rotatable instrument tip that encloses an angled needle that can be extended out from the instrument tip by variable controlled distances, can provide a highly useful device and method, to enable multiple injections of extremely small quantities or liquids, in discrete injection sites that are spaced apart from each other. This can enable injection of larger total quantities of drugs, vectors, or cells, in ways that will be substantially less likely to provoke the types of defensive encapsulation responses that should be avoided.

In view of that information on how and why a certain type of improved CNS injection system performs better than other CNS injectors known in the prior art, it should now become even more clear why a third-generation stereotaxic manipulator system as disclosed herein, which provides a new form of precise angular control and which enables new controlled-angle approaches to targeted brain regions in a mouse or rat, is a substantial improvement over prior known stereotaxic manipulators that cannot provide comparable precision angular control.

However, it also should be noted that this illustration offers just one example of how and why precise angular control can be highly valuable, in invasive neurological tests and procedures. Now that this new and improved system has become available, those skilled in neurological research on small animals will recognizes other valuable uses, advantages, and options for this improved system.

Thus, there has been shown and described a new and useful device and method for providing improved “third generation” digital stereotaxic holders, with precision control over angling options, and with a “fine drive” system that allows ultra-precise control over motion along one or more axes. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A digital stereotaxic manipulator, comprising:
(a) orthogonal components having a design and construction that provide orthogonal control, by means of rotatable threaded shafts, over dorsal/ventral and medial/lateral movement of a manipulator component to which an instrument can be affixed, and having electronic components mounted thereon which can emit electronic signals that can be processed to indicate relative position of manipulator components that move in dorsal/ventral and medial/lateral directions; and,
(b) at least one rotary encoder, capable of measuring angular displacement of at least one orthogonal component of said digital stereotaxic manipulator, and capable of emitting electronic signals that can be processed to indicate angular displacement of said orthogonal component,
and wherein the digital stereotaxic manipulator is sized for use in research on non-human animals.

2. The digital stereotaxic manipulator of claim 1, wherein the orthogonal components comprise:
(a) a vertical arm assembly, which can be used to control dorsal/ventral movement of a traveling block; and,
(b) a horizontal arm assembly which is carried by the traveling block, and which provides an instrument attachment device at one end of said horizontal arm;
wherein a rotary encoder is mounted on said digital stereotaxic manipulator in a manner that enables measurement of angular displacement of said vertical arm assembly, about a vertical axis.

3. The digital stereotaxic manipulator of claim 1, wherein the orthogonal components comprise:
(a) a vertical arm assembly, which can be used to control dorsal/ventral movement of a traveling block; and,
(b) a horizontal arm assembly which is carried by the traveling block, and which provides an instrument attachment device at one end of said horizontal arm;
wherein a rotary encoder is mounted on said digital stereotaxic manipulator in a manner that enables measurement of angular displacement of said vertical arm assembly about a horizontal axis.

4. The digital stereotaxic manipulator of claim 1 which comprises at least two rotary encoders, wherein:
(a) at least one rotary encoder is mounted on said digital stereotaxic manipulator in a manner that enables measurement of angular displacement of said vertical arm assembly about a horizontal axis; and,
(b) at least one rotary encoder is mounted on said digital stereotaxic manipulator in a manner that enables measurement of angular displacement of said vertical arm assembly about a vertical axis.

5. The digital stereotaxic manipulator of claim 1, wherein at least one first rotatable threaded shaft which controls movement of an instrument affixed to said manipulator is provided with a first gear mounted on said shaft that interacts with a second gear on a second rotatable shaft, in a manner that allows rotation of the second shaft and second gear to control rotation of the first rotatable threaded shaft.

6. The digital stereotaxic manipulator of claim 5, wherein the first rotatable threaded shaft is provided with a radial gear affixed to said first shaft, and the second rotatable shaft is provided with a helical gear affixed to said second shaft.

7. The digital stereotaxic manipulator of claim 1, wherein the manipulator is also packaged with a programmable electronic display device that can display orthogonal coordinates for an instrument that is affixed to the manipulator during a procedure.

8. The digital stereotaxic manipulator of claim 7, wherein the manipulator is packaged with a programmable electronic display device that can display orthogonal coordinates that have been mathematically adjusted to account for angular displacement of at least one manipulator component about a horizontal or vertical axis.

9. The digital stereotaxic manipulator of claim 7, wherein the programmable electronic display device can display orthogonal coordinates with a resolution of 10 microns or smaller.

10. A digital stereotaxic manipulator, comprising orthogonal components having a design and construction that provide orthogonal control, by means of rotatable threaded shafts, over movement of an instrument that is affixed to said manipulator, and having electronic components mounted thereon which can emit electronic signals that can be processed to indicate relative motion of manipulator components that move in dorsal/ventral and medial/lateral directions,
wherein at least one first rotatable threaded shaft which controls movement of an instrument is provided with a gear mounted on said shaft that interacts with a second gear on a second rotatable shaft, in a manner that allows rotation of the second shaft and second gear to control rotation of the first rotatable threaded shaft,
and wherein the digital stereotaxic manipulator is sized for use in research on non-human animals.

11. The digital stereotaxic manipulator of claim 10, wherein the first rotatable threaded shaft is provided with a radial gear affixed to said first shaft, and the second rotatable shaft is provided with a helical gear affixed to said second shaft.

12. The digital stereotaxic manipulator of claim 10, wherein the manipulator is also provided with at least one rotary encoder mounted on the manipulator in a manner that renders it capable of measuring angular displacement of at least one orthogonal component of said digital stereotaxic manipulator, and capable of emitting electronic signals that can be processed to indicate angular displacement of said orthogonal component.

13. The digital stereotaxic manipulator of claim 10, wherein the manipulator is packaged with a programmable electronic display device that can display orthogonal coordinates that have been mathematically adjusted to account for angular displacement of at least one manipulator component about a horizontal or vertical axis.

14. The digital stereotaxic manipulator of claim 13, wherein the programmable electronic display device can display orthogonal coordinates with a resolution of 10 microns or smaller.

15. An electronic display device for use with a stereotaxic manipulator, comprising:
a. at least one electronic connector, which enables the display device to be coupled to, and to receive electronic data from, at least two linear reader heads and at least one rotary encoder, all mounted on a single digital stereotaxic manipulator;
b. a display screen that enables orthogonal coordinates to be displayed in digital form with a resolution of 10 microns or smaller;
c. means for enabling commands to be entered into the display device, by a human operator; and,
d. internal electronic circuitry which is designed and capable, when properly loaded with programming instructions, of carrying out each of the following operations:
(1) receiving electronic signals from a stereotaxic manipulator having at least two linear reader heads and at least one rotary encoder;
(2) processing those signals to convert them into orthogonal coordinates that have been mathematically adjusted to account for angular displacement of one or more manipulator components about a horizontal or vertical axis; and,
(3) displaying mathematically-adjusted orthogonal coordinates.

16. The electronic display device of claim 15, which is also designed and capable, when: properly loaded with programming instructions, of carrying out each of the following operations:
a. setting all orthogonal values to zero, under control of an operator, at a selected moment during a procedure; and,
b. displaying subsequent values as orthogonal distances relative to a location that was established as a zero-coordinate location by the operator.

17. The electronic display device of claim 15, which also comprises at least one means for loading software into the display device.

18. The electronic display device of claim 15, which also comprises at least one input/output port that is designed to be capable of transferring data to a computer during a procedure using an animal.
